(19) United States
(12) Patent Application Publication
(10) Pub. No.: US 2013/0325007 A1
(43) Pub. Date: Dec. 5, 2013

(54) SEGMENTED INTRAMEDULLARY IMPLANT

(71) Applicants: Mordechay Beyar, Caesarea (IL); Oren Globerman, Kfar-Shemaryahu (IL)

(72) Inventors: Mordechay Beyar, Caesarea (IL); Oren Globerman, Kfar-Shemaryahu (IL)

(73) Assignee: Carbofix Orthopedics Ltd., Herzlia Pituach (IL)

(21) Appl. No.: 13/985,048

(22) PCT Filed: Feb. 13, 2012
(86) PCT No.: PCT/IB12/50645

§ 371 Date: Aug. 13, 2013

Related U.S. Application Data
(60) Provisional application No. 61/442,248, filed on Feb. 13, 2011.
(60) Provisional application No. 61/443,308, filed on Feb. 16, 2011.
(60) Provisional application No. 61/486,280, filed on May 15, 2011.

(30) Foreign Application Priority Data
Jun. 7, 2011 (IB) PCT/IB2011/052468

(51) Int. Cl. A61B 17/72

(52) U.S. Cl. CPC A61B 17/72
USPC 606/62

(57) ABSTRACT

A segmented intramedullary nail having an implantation state in which the segments are relatively moveable to permit conformation to the curvature of a bone during implantation, and a post-implantation state in which the segments are not relatively moveable, and in which the segments retain the configuration of the curvature of the bone. The nail may be radiolucent and/or contain radiopaque markings. It may be provided independently or as part of a kit with an implantation and a tightening tool. A cap and/or one or more sleeves may be provided to prevent bone in-growth at a point of coupling to an insertion tool and/or at the interface between segments.

### RELATED APPLICATION/S

This application is based on and claims priority to U.S. Provisional Application, Ser. No. 61/442,248, filed 13 Feb. 2011, the contents of which are incorporated by reference as if fully set forth herein.

This application also is based on and claims priority to International Application IB2011/052468, which in turn, is based on and claims priority to U.S. Provisional Application, Ser. No. 61/344,182, filed 7 Jun. 2010, U.S. Provisional Application, Ser. No. 61/443,308 filed 16 Feb. 2011, and U.S. Provisional Application, Ser. No. 61/486,280filed 15 May 2011.

The contents of the foregoing applications and any publications mentioned herein are hereby incorporated by reference as if fully set forth.

### FIELD OF INVENTION

The present invention, in some embodiments thereof, relates to bone implant fixation technology, and more particularly, but not only, to intramedullary fracture fixation and bone strengthening devices In some embodiments, the invention also relates to methods of long bone fixation and strengthening, and to kits including implants and one or more insertion tools.

### BACKGROUND OF THE INVENTION

Intramedullary fixation provides an alternative to open reduction and fixation of a variety of fractures. The objective of this closed technique as compared to open techniques is to provide fixation with minimal trauma, reduced risk of infection, and reduced blood loss.

Intramedullary nailing of longer bones was introduced in 1939 in Germany by Gerhard Küntscher. Since then, intramedullary fixation for long bone fractures has been an accepted technique in the armamentarium of the orthopedic surgeon for more than 4 decades. Over the years the indications expanded and new implant designs have been introduced. For example, intramedullary implants may be used to strengthen long bones in the treatment of fractures or pending fractures, but the operation technique has not greatly changed. In the forearm bones, the concept of an implant traversing the full medullary canal was used even before it was used in the femur. Schöne was the first to use medullary fixation in both the radius and ulna in 1913, using silver rods.

In general, intramedullary nails are rod-shaped, rigid, devices that may be secured (interlocked) to the bone using one or more locking elements, such as transverse screws at one or both nail ends.

Two issues associated with intramedullary nailing of long bone fractures, and especially in certain bones (e.g., the forearm bones—radius and ulna) are visibility of the fracture line during and after operation, and implant rigidity during its insertion into the medullary canal, limiting the choice of bone entry point, and resulting, in certain cases in additional bone fracture and harm to soft tissue (e.g., muscles, tendons).

Therefore, in certain situations, intramedullary nailing is not the treatment of choice. One reason for this is that conventional intramedullary fixation devices diminish the possibility for good fracture alignment. In the case of the forearm bones, functional integrity is tightly connected with proper cooperation between ulna and radius in the distal and proximal radioulnar joint. Incongruity in these joints can be caused by misalignment of either the ulna or radius concerning length, axis, and torsion. This can result in corruption of forearm rotation and can limit the function in adjacent joints.

Conventional treatment methods for fixation of forearm bones include the use of intramedullary pins and wires or bone plates. Using pins and wires requires shaping the implants prior to insertion into the medullary canal to comply with its curvature. The use of bone plates requires wide exposure of the fracture region. A device that will enable minimally invasive surgery while providing flexibility in the choice of bone entry point, device compliance with the shape of the intramedullary canal, and not compromising implant rigidity once implanted has advantages in these respects.

Intramedullary fracture fixation devices that enable implant flexibility during insertion and rigidity while implanted have been suggested in the past. Some examples are found in U.S. Pat. No. 5,879,352, U.S. Pat. No. 7,785,325, WO 2008/064346, WO 2009/152270 A1, and WO 2009/152272 A1, but these have some limitations. Other relevant prior art includes published International Application WO 1993/13713 which discloses a segmented instrument for use in abdominal surgery having flexible and rigid states.

### SUMMARY OF THE INVENTION

Among the objects of some embodiments of the present invention are to provide bone implants, implantation systems and bone fixation and strengthening methods having various desirable features.

According to an aspect of some embodiments of the present invention there is provided an intramedullary nail in the form of an elongated structure including proximal and distal end segments and a coupling arrangement between the segments, in which the coupling arrangement provides a first implantation state in which the proximal and distal segments are relatively moveable to allow conformity to curvature of a bone, and a second post-implantation state in which the segments are locked together to provide a substantially rigid structure that substantially follows the curvature of the bone which it assumed during implantation.

In some embodiments, the segments have substantially planar proximal and distal end surfaces that lock together with the end surfaces of adjacent segments when the device is in the rigid state, and remain in the relative positions assumed in the flexible state.

In some embodiments, the segments have substantially smooth end surfaces or alternatively, end surfaces that are treated to increase the friction between the surfaces.

In some embodiments, the end surfaces have features that lock together with complementary features on the end surfaces of adjacent segments when the implant is in the post-implantation state. In some embodiments, the features are teeth, or projections that are harder than the material of which the segment is made.

In some embodiments, the distal segment includes a cutting surface to allow reaming of the intramedullary canal during insertion.

In some embodiments, the distal end of the distal segment is conical. Alternatively, it is rounded.

In some embodiments, there are one or more intermediate segments between the proximal and distal end segments.

In some embodiments, the segments are rotatable relative to each other in the implantation state.

In some embodiments, the coupling arrangement includes a tightening element operable to transform the implant from the implantation state to the post-implantation state.

In some embodiments, the tightening arrangement draws the distal and proximal segments toward each other in the post-implantation state.

In some embodiments, the segments are cannulated and the tightening element is a flexible rod that extends through the cannulations, and is connected to the distal and proximal segments.

In some embodiments, the tightening element is a solid rod, made of stainless steel, or titanium, or a titanium alloy, or other implantable metal.

In some embodiments, the tightening element is made of oriented fibers or interwoven cable, or fibers embedded within a matrix made of implantable material. The matrix may be polyetheretherketone (PEEK), polyetherketoneketone (PEKK), or other implantable polymer.

In some embodiments, the fibers are carbon and/or ultra high molecular weight polyethylene (UHMWPE).

In some embodiments, the segments are cannulated and the long axis of the cannulation is collinear with the long axis of the segment. Alternatively, the long axis of the cannulation is eccentric to the long axis of the segment. The long axis may be parallel to or not parallel to the long axis of the segment.

In some embodiments, different segments may have differently oriented cannulation. In some embodiments, the rod is solid, or alternatively, cannulated and configured to receive a guide wire.

In some embodiments, the rod is press-fitted into a bore in the distal end segment. Alternatively, the rod may be threaded into the distal end segment.

In some embodiments, the rod is threaded at its proximal end and engages with 25 threads in the proximal end segment.

In some embodiments, the threads in the proximal end segment are comprised in a nut fixed to the proximal end segment.

In some embodiments, the distal segment has a cutting edge.

In some embodiments, the distal segment has a conical end or a rounded end.

In some embodiments, the cross-sectional contours of the proximal and/or distal end segments are formed of multiple polygonal or curved elements. Alternatively, the contours may be round or elliptical.

In some embodiments, the cross sections of some or all the elements are slotted.

In some embodiments, different segments of the device have different cross sectional shapes.

In some embodiments, the transverse external dimensions of the segments are constant along the entire length of the device. Alternatively, the transverse external dimensions of the device segments vary from segment to segment, or along a segment.

In some embodiments, the tightening element transforms the nail from its implantation state to its post-implantation state by moving the segments apart.

In some embodiments, the rod is formed of a metal, a polymer, or a fiber-reinforced polymer composite.

In some embodiments, the rod is formed of one of stainless steel, titanium, or a titanium alloy, or alternatively, of one or more polymers, for example, PEEK and/or PEKK.

In some embodiments, the rod is formed of oriented fibers or an interwoven fiber cable, or reinforcing fibers embedded in a polymer matrix.

In some embodiments, the fibers are formed of carbon, or ultra high molecular weight polyethylene.

In some embodiments, the tightening element includes a coupling at its proximal end configured to receive a tightening tool.

In some embodiments, the coupling is a solid or cannulated threaded bolt.

In some embodiments, the nail includes holes for receiving locking elements to fix the implanted nail in the bone.

In some embodiments, the holes are located externally to the tightening element. Alternatively, at least some of the holes extend through the tightening element.

In some embodiments, two adjacent holes are paired.

In some embodiments, the paired holes are located circumferentially displaced by 90 degrees.

In some embodiments, the interface between the segments of the device allows flexibility in a single plane when the implant is in the flexible state.

In some embodiments, the interface between the segments comprises projections on one surface and complementary recesses on the adjacent surface.

In some embodiments, the interface between the segments comprises pins on one surface and complementary holes on the adjacent surface.

In some embodiments, the interface between the segments comprises ribs on one surface and complementary slots on the adjacent surfaces.

In some embodiments, the interface between the segments of the device allows flexibility in one or more planes when the implant is in the flexible state.

In some embodiments, the recesses are wider than the projections.

In some embodiments, portions of the projections are curved and portions of the complementary recesses match the curvature, and further projections that are not curved and lock the segments together in the post-implantation state.

In some embodiments, the further projections and complementary recesses are polygonal.

In some embodiments, the proximal segment includes a coupling element for connection of the implant to an insertion and/or a tightening tool.

In some embodiments, the coupling element is threaded, or includes one or more elements to provide for desired alignment of the tool and the implant, and to allow transfer of rotational movement.

According to an aspect of some embodiments of the invention, there is provided a long-bone fixation implant in the form of a segmented intramedullary nail having an implantation state in which the segments are relatively moveable and a post-implantation state in which the segments form a substantially rigid structure that substantially conforms to a curvature of a bone in which it is implanted, wherein the nail is at least partially formed of a radiolucent material.

In some embodiments, the nail is formed of a radiolucent ceramic, for example, zircona, or a polymeric material, for example, PEEK, PEKK, and PAEK.

In some embodiments, the polymeric material includes reinforcing fibers, formed, for example, of chopped carbon.

In some embodiments, the implant is formed of one or more injection molded polymeric segments.

In some embodiments, the nail includes one or more intermediate segments formed of a radiolucent material between proximal and distal end segments formed of metal, for example, titanium, or stainless steel.

In some embodiments, a proximal segment, a distal segment, and one or more intermediate segments are all formed of a radiolucent material.

In some embodiments, the nail includes one or more radiopaque markings to allow visualization under imaging.

In some embodiments, the radiopaque markings are comprised of markers located along an axis of one or more of the segments, and/or one or more markers located at the periphery of one or more of the segments, and/or one or more rings located around the circumference of the segment, and/or dots embedded within the segment.

In some embodiments, in markings are formed of one of tantalum, platinum, gold and tungsten.

In some embodiments, the proximal and distal end segments are formed with one or more holes for receiving locking elements to fix the nail within the bone, and radiopaque markings defining the positions of the holes.

According to an aspect of some embodiments of the invention, there is provided a bone fixation system that includes an intramedullary nail as described herein, an insertion tool that cooperates with a coupling element on the nail to insert the nail into the medullary canal of a bone and a tightening tool for operating the tightening element to transform the nail between its implantation state and its post-implantation state.

In some embodiments, there is also provided a cap configured to engage within the coupling to protect the coupling from bone in-growth, and/or to allow nail removal.

In some embodiments, one or more flexible sleeves are provided to cover at least part of the implant, to prevent tissue in-growth into the areas of connection of the device segments.

In some embodiments, the insertion tool is a single use device.

In some embodiments, the insertion tool is preassembled onto the implant and is packed together with the implant.

In some embodiments, the insertion tool is a multiple use device.

According to an aspect of some embodiments of the invention, there is provided a tool for implanting a bone nail as described herein having the tool comprising a cannulated insertion handle that engages the nail for insertion into the medullary canal of a bone and a tightening tool that extends through the cannulation to engage the tightening element of the nail to transform the nail between the implantation and post implantation states.

According to an aspect of some embodiments of the invention, there is provided a method for long bone fixation in which a segmented bone nail is provided that has an implantation state in which the segments are movable relative to each other, and post-implantation state in which the segments are substantially rigidly connected to each other, inserting the bone nail in the implantation state into the medullary canal of a bone, whereby the nail conforms to curvature of the medullary canal during implantation; and transforming the nail from the implantation state to the post-implantation state, in which the conformity to the curvature of the medullary canal is retained.

In some embodiments, before transformation to the post-implantation state, one or more locking elements are inserted through holes in a distal segment of the nail to anchor nail in the bone.

In some embodiments, after transformation of the nail to the post-implantation state, one or more locking elements are inserted through holes in the proximal segment to further anchor the implant in the bone.

In some embodiments, the nail is transformed to its post-implantation state by operating the tightening element to draw the segments together.

In some embodiments, the tightening element is rotatable, and is operated by coupling a proximal end of the nail to a transformation tool and rotating the tool.

In some embodiments, the distal segment includes a cutting element, and, during implantation, the nail is rotated to ream the medullary canal.

According to an aspect of some embodiments of the invention, there is provided a method for removing a long bone fixation nail as described herein, that involves, exposing the implantation site, removing any locking elements to anchor the nail to the bone, transforming the nail from the post-implantation state to the implantation state and pulling the nail out of the bone.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE FIGURES

Some exemplary embodiments of the invention will be further described with reference to the accompanied drawings, in which:

FIGS. 1A and 1B are schematic illustrations of a nail, in accordance with some embodiments of the present invention, in a straight state and in a flexed state;

FIGS. 2A and 2B are schematic illustrations of different designs of a cross section of the nail shown in FIG. 1, in accordance with some embodiments of the present invention;

FIGS. 3A and 3B are schematic illustrations of different designs of a cross section of the nail shown in FIG. 1 with a tightening element in place (in a rigid state), in accordance with some embodiments of the present invention;

FIGS. 4A-4D are schematic illustrations of exemplary designs of nail body segments, in accordance with same embodiments of the present invention;

FIGS. 5A-5C are schematic illustrations of exemplary designs of nail distal segment, in accordance with same embodiments of the present invention;

FIG. 6 is a schematic illustration of an insertion assembly, in accordance with some embodiments of the present invention, where the insertion assembly is connected to a nail as shown in FIG. 1;

FIG. 7A is a schematic illustration of an insertion handle in accordance with some embodiments of the present invention;

FIG. 7B is a schematic illustration of a tightening tool and connected to an optional; tightening element in order to change the implant state from flexible to rigid;

FIG. 8 is a schematic illustration of several components of an insertion assembly as shown in FIG. 6, in accordance with some embodiments of the present invention;

FIG. 9 is a flow diagram showing an exemplary method of inserting a bone nail according to some embodiments of the invention; and

FIG. 10 is a flow diagram showing an exemplary method of removing a bone nail according to some embodiments of the invention

### DETAILED DESCRIPTION OF SOME EMBODIMENTS OF THE INVENTION

### Introduction

The present invention, in some embodiments thereof, relates to bone implant fixation technology, and more particularly to intramedullary fracture fixation and bone strengthening devices. In some embodiments, the invention also relates to methods of long bone fixation and strengthening, and to kits including implants and one or more insertion tools.

Generally, some embodiments pertain to devices for fixation of fractures or pending fractures or preventing of fractures that have a flexible implantation state in which the implant can conform to the longitudinal path of a bone in which the device is being implanted, and a rigid post-implantation state to which it is transformable, in which the implant forms a rigid structure that remains in the configuration assumed during implantation.

In some embodiments, the implant includes proximal and distal end segments, and optionally, one or more intermediate segments, and a tightening element operable to transform the implant from the implant state to the post-implant state. In some embodiments, the tightening element is fixedly connected to the distal end segment, and threadedly connected to the proximal end segment, whereby rotation of the tightening element in one direction draws the segments closer together to transform the implant from the implantation state to the post-implantation state. Rotation in the other direction pushes the segments apart, thereby transforming the implant from the post-implantation state to the implantation state, for example if the implant is to be removed.

The segments are cannulated to receive the tightening element.

Optionally, at least part of the device is configured to be rotatable during insertion into the medullary canal.

In some embodiments, distal and proximal end surfaces of adjacent segments are configured to be angularly moveable relative to each other to permit the nail to conform to curvature of the bone during implantation. When transformed to the post-implantation state, the end surfaces lock together and remain in the relative positions assumed in the flexible state.

Optionally, in some embodiments, the end surfaces are relatively smooth or alternatively roughened. Further alternatively, the ends of the segments include complementary features that are relatively moveable with one or more degrees of freedom in the implantation state, but lock together in the post-implantation state. Exemplary structures are described below.

Some embodiments pertain to implants that are formed of radiolucent material to allow visibility of the fracture, during and/or after implantation. Such implants may allow easier and more accurate fracture reduction than other implants which block view of the fracture line.

In some such embodiments, the segments of the implants are formed of any implantable i.e., bio-compatible radiolucent material. Such materials include, but are not limited to, ceramics, polymers (including, but not limited to, polyetheretherketone (PEEK), polyetherketoneketone (PEKK), polyaryletherketones (PAEK)), composites including, but not limited to chopped carbon fiber-reinforced polymers, and any combination of such materials.

Optionally, in some embodiments, the distal end segments of implants as described herein are formed to cut the bone during implantation. In such embodiments, the distal end segments may be formed of an implantable metal, including, but not limited to, titanium, titanium alloys, or stainless steel. Other parts except for those that will be positioned in the vicinity of the fracture may optionally be formed of metal as well.

Optionally, in some embodiments as described herein, the device segments are fabricated by injection molding of polymers with or without fibers (for example, randomly oriented chopped carbon fiber reinforced PEEK).

Optionally, in some embodiments as described herein, radiopaque material is incorporated into the radiolucent segments to allow the implant to be clearly visualized using imaging devices such as fluoroscopy, X-ray, etc. during the surgical process.

Optionally, radiopaque markings, for example, in the form of one or more elongated wires may be located along part or the entire length of the segments, for example, parallel to the longitudinal axes of the segments. Circumferential markers formed of curved wires or rings may alternatively or additionally employed. As a further alternative, radiopaque dots may embedded within the segments to mark the outline of the segments. Optionally, radiopaque markers such as curved wires, rings, or dots can be placed around holes in the proximal and distal end segments that receive elements, for example, standard bone screws, pins, or Kirschner wires (K-wires), for anchoring the nail in the bone. Optionally, the radiopaque materials may, without limitation, include tantalum, platinum, gold and tungsten. Typically, the markings are molded into the implant.

Typically, the cross-sections of the proximal and distal end segments are continuous, for example circular or elliptical. Optionally, the cross-section can be polygonal. A cross-section formed of flat sections may facilitate inserting K-wires as anchoring devices or drilling for fixation screws or pegs.

In some embodiments, the transverse external dimensions of the segments (e.g., the diameter in the case of a cylindrical nail) are constant along the entire length of the segments. Optionally, the external dimensions of the segments may vary from segment to segment, or along a segment, to allow usage, for example, in bones with tapered intramedullary canals.

As mentioned above, the interfaces between the segments are angularly moveable relative to each other in the implantation state. Optionally, interface configurations include smooth or generally planar surfaces, or generally planar surfaces that are treated to increase the friction between them in the post-implantation state. For example, one or both surfaces may be treated, for example by sand blasting.

In some embodiments, projections, for example, teeth or ribs are provided on one surface which mate with complementary receptacles on the adjacent surface. Optionally, the surfaces provide ball-and-socket type connections. Optionally, associated with the ball and socket connection, features are included that can lock together in the implantation state to transmit rotation, and which also lock together in the post-implantation state.

As a further option, at least one of the surfaces may incorporate sharp protrusions made of implantable materials that are harder than the material of which the segment is made.

In an embodiment of the present invention, the distal end segment provides a cutting element to allow reaming during insertion of the device into the medullary canal. Alternatively or additionally, the distal segment has a conical or a rounded end to facilitate penetration of the medullary canal. Optionally, the segments may have longitudinal grooves to allow material removed by reaming to be dissipated.

In some embodiments of the present invention, the proximal segment provides means for connection of the implant to instruments (for example, but not limited to, an (optionally rotatable) insertion tool. In an exemplary embodiment the connection detail provides for a thread for the connection of instrument to the device. In another exemplary embodiment, the proximal end of the proximal section has an asymmetrical, for example, “crown”-like, configuration for alignment with the installation tool and to allow transfer of rotational movement.

In some embodiments of the invention, the distal and/or proximal segments incorporate wing-like components that, upon deployment, penetrate the bone from inside the medullary canal, to anchor the device to the bone. In some embodiments., the proximal and/or distal segments include holes configured to receive fixation elements, for example, K-wires, pins, or screws, that are inserted into the bone following insertion of the device into the medullary canal, to anchor the device to the bone.

Optionally, the deployable fixation elements are attached to the nail segments before implantation, for example, along the perimeter of the segment(s). Alternatively, the deployable fixation elements are inserted through cannulations in the segments after insertion of the device into the medullary canal.

In some embodiments, the wing-like anchoring components and/or deployable fixation elements are installed and/or deployed using the tightening element. In some embodiments, installation and/or deployment is facilitated by a guiding accessory on an insertion tool.

In an embodiment the holes are located in one plane transverse to the long axis of the device. In another embodiment the holes are located at several planes transverse to the long axis of the device, with at least two opposing holes aligned on the same axis, to allow insertion of the locking element. In an embodiment the central axis of the holes intersect with the longitudinal central axis of the device. In another embodiment the central axis of the holes is eccentric to the central long axis of the device.

In an embodiment of the present invention any or all of the holes intended for the engagement of locking elements, and/or the holes intended for the deployment of locking elements, and/or the wing-like anchoring elements, are marked with radiopaque markers to facilitate visibility when using imaging devices such as fluoroscopy, X-ray, etc.

In some embodiments, of the present invention, K-wires used for locking the device to the bone, are smooth for their entire length. Optionally, the K-wires are threaded. In some embodiments, smooth K-wires are implanted such that their proximal and/or distal ends protrude from the skin surface. In some embodiments, threaded K-wires are implanted such that they do not protrude from the skin surface.

In some embodiments, the cannulations in segments provided for passage of the tightening device are collinear with the long axis of the segment. In another embodiment, the long axis of the cannulation is eccentric to the long axis of the segment. In yet another embodiment, the long axis of the cannulation is not parallel to the long axis of the segment. In some embodiments, the different segments of the device have differently oriented cannulation.

In an embodiment of the present invention a connection and tightening element (“tightening element”) is provided. In an embodiment, the tightening element is connected to the distal segment of the device at one end and to the proximal segment of the device at its other end.

In an embodiment of the present invention the tightening element is a solid rod, but is sufficiently flexible to allow the segments to conform to the curvature of the bone during implantation, and strong enough in tension to maintain the segments locked together in the post-implantation state. In some embodiments, the rod may be made of any implantable metal, for example, stainless steel or titanium or a titanium alloy.

In some embodiments the tightening element is made of oriented fibers or interwoven cable, or a fiber-reinforced polymer matrix. In some embodiments the fibers are formed, for example, of carbon, ultra high molecular weight polyethylene (UHMWPE), for example, Dyneema®. For embodiments in which the fibers are embedded within a polymer matrix, the polymer forming the matrix may be made of, for example, polyetheretherketone (PEEK), polyetherketoneketone (PEKK),

In an embodiment of the present invention the tightening element is cannulated, to allow insertion of the device into the medullary canal over a guide wire.

In an embodiment, the present invention the tightening element provides, at its proximal end, for connection to an instrument that allows tightening of the implant, to change its state from flexible to rigid. In an exemplary embodiment, such connection detail has the shape of a threaded bolt, either solid or cannulated.

In an embodiment of the present invention the holes for engaging locking elements are located externally to the tightening element. For example, the holes may be located distally of the distal end of the tightening element. Alternatively, the holes a displaced from the axis of the tightening element.

In some embodiments of the invention, a coupling element is provided at the proximal end of the nail to engage an insertion and a tightening tool. Optionally, a “cap” is provided for the coupling element to protect against from bone in-growth, e.g., to allow connection of a removal tool if removal of the nail is desired. In an exemplary embodiment, the cap is bolt with a threaded distal section.

In an embodiment of the present invention, one or more flexible sleeves, formed, for example of an implantable polymer, are placed over the implant, covering at least part of its length, to prevent tissue in-growth at the interfaces of the device segments, to allow the segments to move if the device is returned to the flexible state for removal, as described below.

A device of the type disclosed herein with reference to some embodiments of the invention may be sized and configured for use in fixation of bones in various regions of the body, especially bones with pronounced curvature, including, but not limited to the radius, ulna, clavicle, metacarpals, and metatarsals. Optionally, the device can be used generally straight bones such as the humerus, tibia, fibula and femur The device is provided in a range of cross-sectional dimensions, and in a range of lengths, as discussed in detail below.

The device disclosed herein may be used as a stand-alone fracture fixation device, or may be used in combination with cast, for either the entire, or part of, the implantation period.

Embodiments of the present invention pertain also to implantation systems including implants, and implant and/or removal tools (“insertion tool”), for the device.

In some embodiments, the insertion tool functions conventionally for introduction of the device into medullary canal. In another embodiment the insertion tool is configured to permit tapping it with a hammer to facilitate insertion into the medullary canal.

In some embodiments, the insertion tool is configured to rotate the device, to permit reaming of the medullar canal during installation, where such feature is provided, and to assist in device advancement through the medullary canal.

In some embodiments, the insertion tool provides also for operation of the tightening element to transform the device from the implantation state to the post-implantation state, or vice versa, for device removal. For this purpose, a portion of the tool may be rotatably located in a cannulation of the main body of the tool.

In an embodiment of the present invention the insertion tool is provided with an attachment that provides as a drill guide for drilling holes for locking elements, and/or as a guide for insertion of locking elements through holes provided in the proximal segment of the device.

The insertion tool may be discarded after a single use or appropriately sterilized for reuse. Optionally, the insertion tool is preassembled onto the implant and is packed together with the implant.

### Exemplary Embodiments of A Bone Nail:

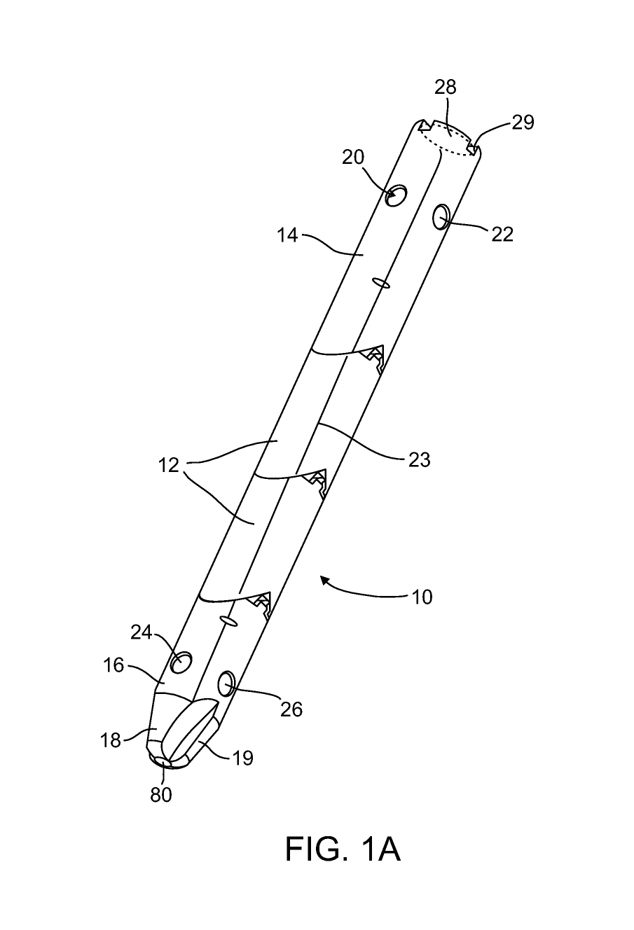

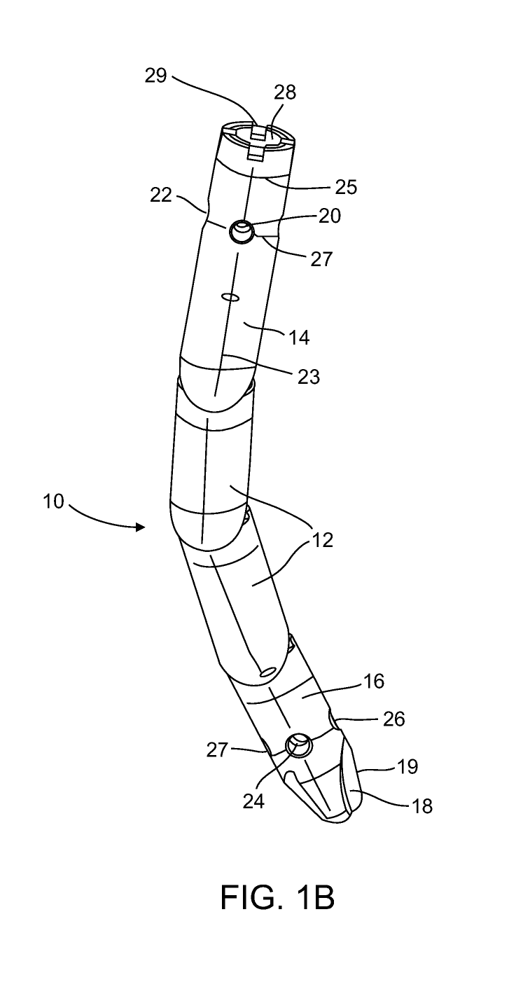

Turning now to the drawings, FIGS. 1A, 1B, 2A, 2B, 3A and 3B illustrate an exemplary implant in the form of an intramedullary nail 10 according to some embodiments of the invention. For illustrative purposes, intramedullary nail 10 is shown as an implant for use in fixation of fractures of the radius and ulna bones, but it should be kept in mind that nails may also be dimensioned and/or shaped for fixation of other curved bones, for example, the clavicle, metacarpals, and metatarsals, or for straight bones such as the Tibia, Fibula and Femur. Also, use for strengthening bones for example, for preventing incipient fractures, is also within the scope of the invention.

Nail 10 is comprised of a proximal end segment 14, a distal segment end 16, and intermediate body segments 12. For fixation of the radius or ulna, two intermediate segments are shown for purposes of illustration.

The number and dimensions of the segments generally depends on the size of the particular bone to be treated, and its curvature.

Generally, nails may be provided with segments that range in diameter from about 3.5 mm-8 mm (for segments having circular cross-sections), and range in length from about 180 mm-280 mm. As will be appreciated, nails at the lower ends of the size ranges will be used for short bones, for example, metacarpals.

As will be appreciated, shorter segments will be used for fixation of shorter bones. Short segments can also be advantageous for treating bones having substantial curvature and to facilitate entry into the bone during implantation. Optionally, for ease of insertion, the proximal and/or distal end segments may be shorter than the intermediate segments.

Nail 10 is advantageously formed of a radiolucent material, for example, a ceramic or polymeric material to permit radiographic imaging during implantation. A suitable ceramic material can be, for example, zirconia.

Suitable polymeric materials include polyetheretherketone (PEEK), polyetherketoneketone (PEKK), polyaryletherketones (PAEK)), composites including, but not limited to chopped carbon fiber-reinforced polymers, and any combination of such materials. Optionally, the intermediate segments may be formed of a radiolucent material and the proximal and/or (especially) the distal segments formed of metal, of example, titanium, or stainless steel to facilitate entry into the medullary canal, reaming during insertion and radiographic visualization, and possibly less formation of debris during implantation.

Polymer segments may be fabricated by injection molding, or in any other suitable way.

FIGS. 1A and 1B show exemplary radiopaque markings that may be provided to facilitate radiographic visualization during implantation. Such markings may, for example, be in the form of a line of longitudinal markers 23, for example, wires, embedded below the surface of the segments off the long axis. Markers 23 may extend along the entire length of the segments as shown, or along only part of the length of the segments.

Likewise, markers are shown of all of segments 12, 14, and 16, but may optionally be provided on only some of the segments.

Several lines of longitudinal markers 23 may be provided, for example two lines spaced 180 degrees apart around the periphery of the segments, or four lines spaced 90 degrees apart.

Another useful marker arrangement is shown in FIG. 1B in the form of radiopaque rings 25 and 27 spaced at desired intervals. For example, rings 25 may be provided at the ends of the segments, and rings 27 may be provided to identify the locations of holes provided to receive fixation devices as discussed below, Optionally, instead of complete rings 25 and 27, sections of curved wires forming broken rings (not shown) may provided. In another unillustrated embodiment, the fixation element receiving holes may be delineated by circular or other shaped markers. Optionally, the radiopaque markings may be comprised of tantalum, platinum, gold tungsten or other suitable metals.

As described, for example, in detail below, the interconnection between the segments is designed to provide a flexible implantation state in which the segments are moveable relative to each other, and a substantially rigid post-implantation state (referred to sometimes herein for simplicity as “rigid”) in which the segments are locked to each other in the configuration assumed during implantation, and not relatively moveable. The implantation state permits convenient implantation in a curved bone, while in the post-implantation state, the segments are locked together sufficiently tightly that they remain in a fixed configuration corresponding to the curvature of the bone during the healing process.

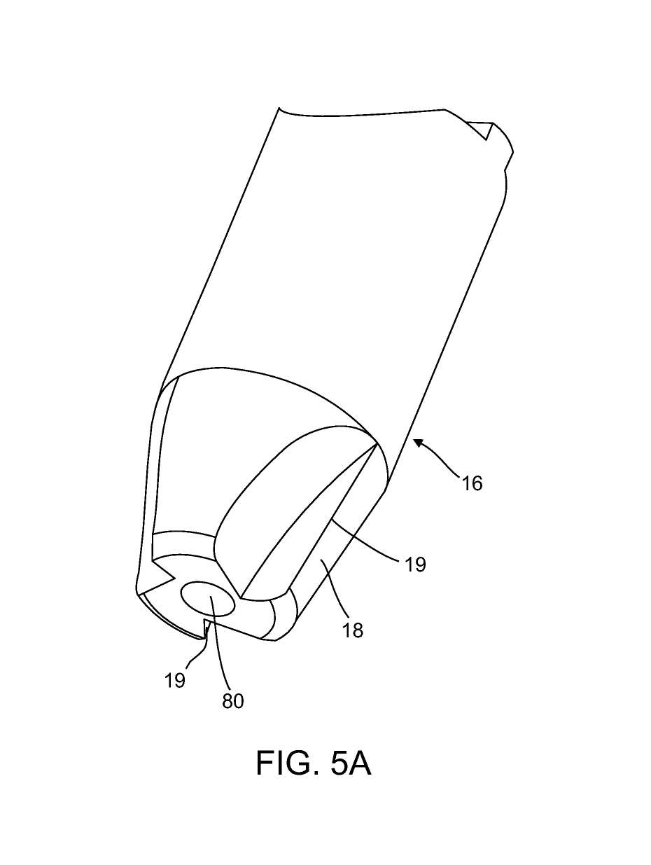

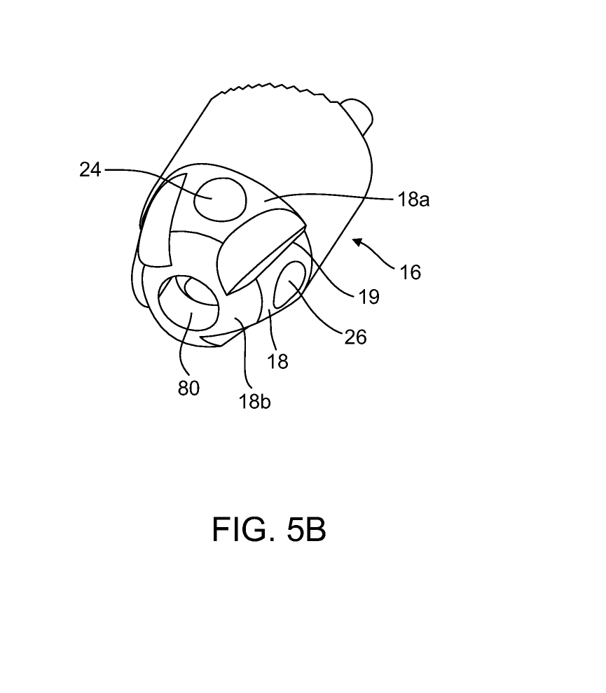

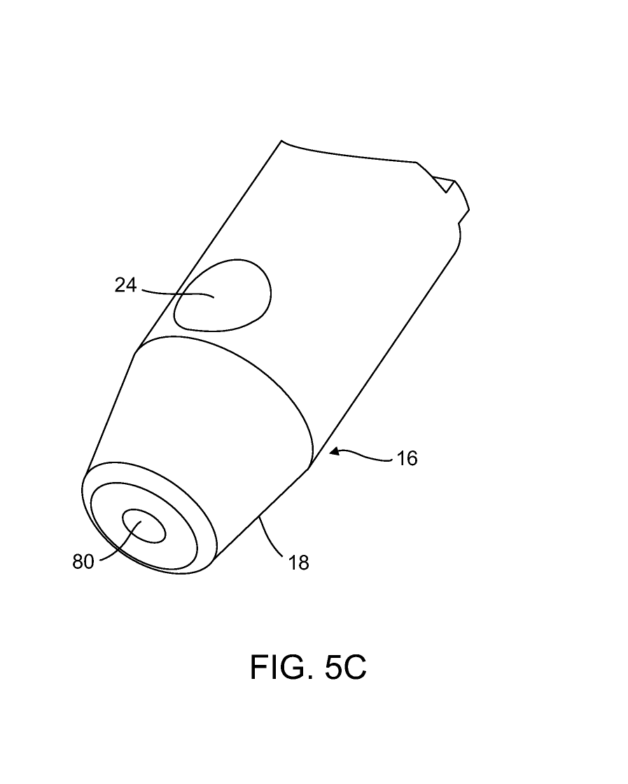

Optionally, in some embodiments, as described in connection with FIGS. 5A and 5B below, tip portion 18 of the distal segment 16 generally rounded or conical to facilitate penetration and entry into the medullary canal. Optionally, tip portion 18 is configured as a cutting head, and includes a cutting edge 19, to allow reaming during nail insertion into the medullary canal.

In the illustrated embodiment, holes 20 and 22 in proximal end segment 14, and holes 24, and 26 in distal end segment 14 are provided to receive fixation elements (not shown) for locking nail 10 into the bone. Suitable fixation elements include conventional bone screws and pegs or K-wires, and the holes re sized accordingly. For example, the diameter of a K-wire used as a locking elements, may be in the following range: 1 mm-1.6 mm, preferably 1.4 mm.

Optionally, the holes are arranged in pairs, with holes 20 and 24 oriented at 90 relative to holes 22 and 26, respectively. Typically, only one of the paired holes will be used, but in general, the exact position of the hole relative to the bone is not known in advance the, and access from all directions for insertion of the locking device may not be possible, due, for example to interference and possible damage other body organs.

Although a paired hole arrangement has been shown, it should be understood that other hole arrangements are possible, (e.g., different number of holes, different orientation, and different shape).

A connection and tightening element (“tightening element”) 32 illustrated as a flexible rod, is located within a cannulation 31 running through the segments of nail 10 to connect the segments and to transform the nail between the implantation and post-implantation states.

In general, rod 32 should be manufactured from material having very high tensile strength. Thus, rod 32 is advantageously formed of longitudinally oriented fibers or alternatively of interwoven cable, for example, carbon, ultra high molecular weight polyethylene (UHMWPE).

Alternatively, rod 32 may be formed of fibers embedded within a matrix made of implantable material, for example, (PEEK), (PEKK), or other implantable polymer. Rods as described above are advantageously resiliently flexible to allow following the curvature of a bone during implantation, but also having sufficient resistance to bending allowing the nail segments to be locked together in the post-implantation state.

As an alternative, rod 32 may be formed of metal, for example, stainless steel such as for example Carpenter 465, titanium and titanium alloy for example Ti4A16V, or alloy like MP35n, again, having very high tensile strength to prevent elongation and release of the tight connection between the segments.

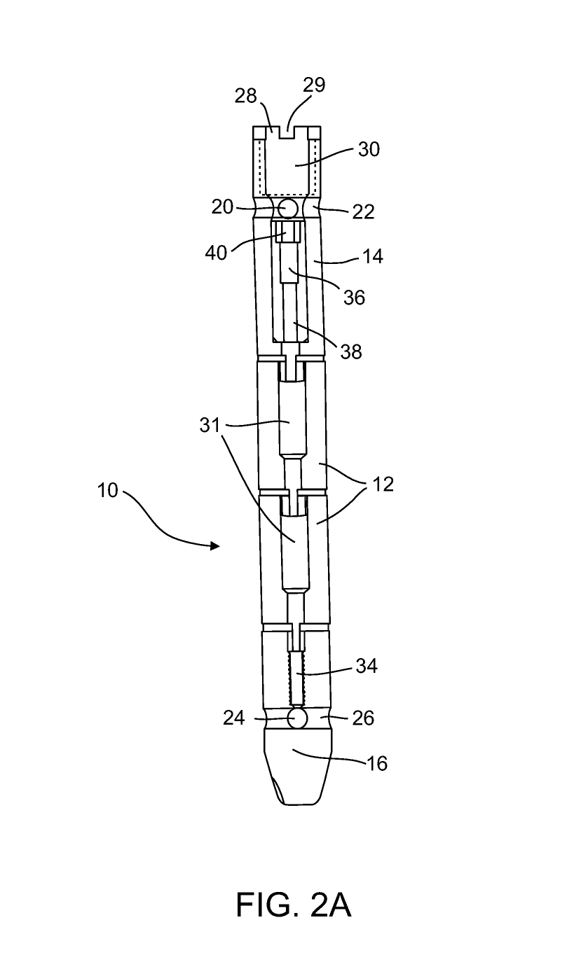

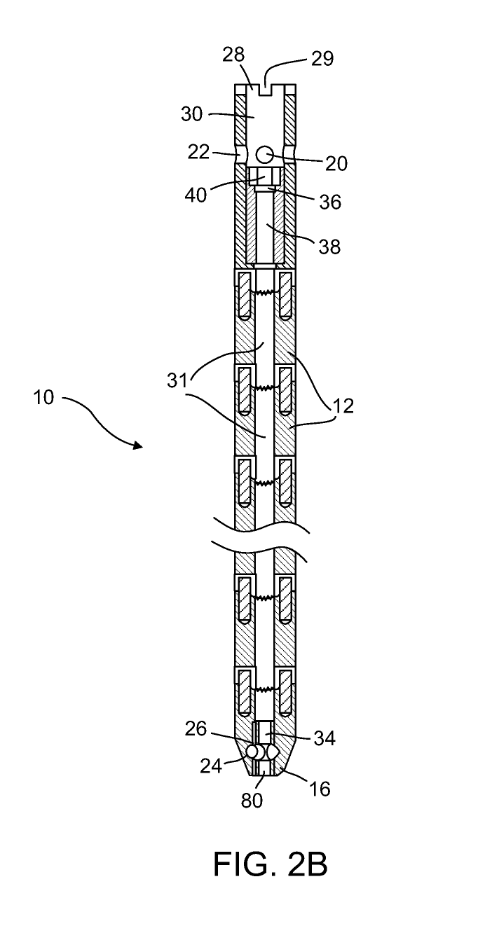

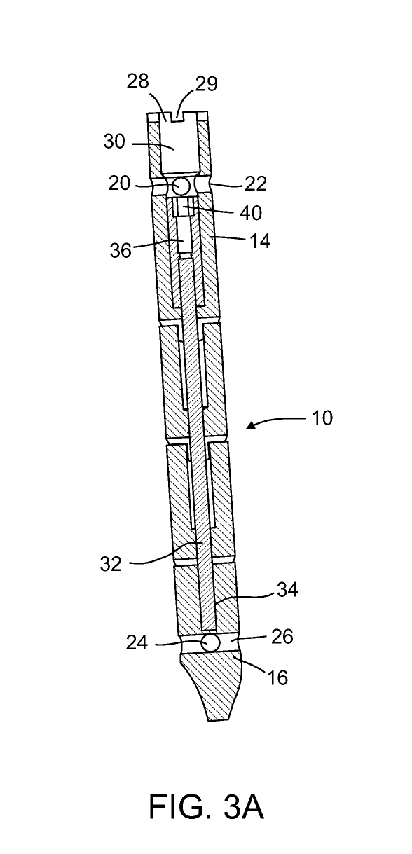

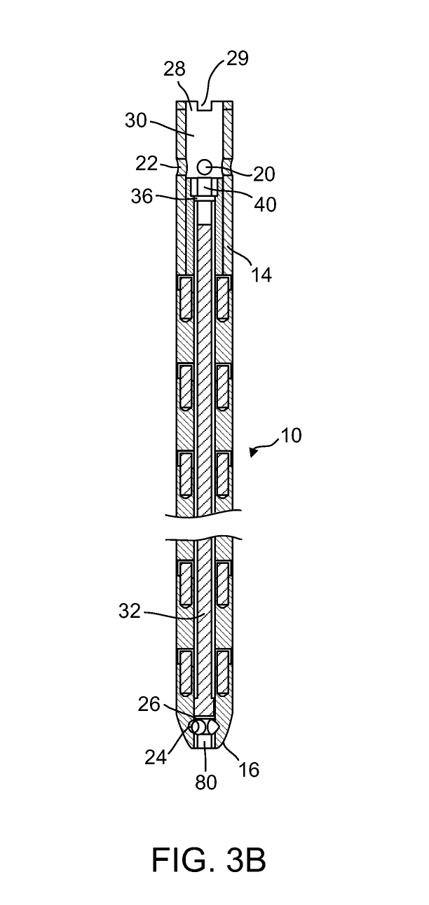

Tightening element 32 is connected to distal segment 16 of nail 10 at one end 34, and the proximal segment 14 at its other end. In the embodiments illustrated in FIGS. 2A, 2B, 3A and 3B, a threaded connection between the proximal end segment and the tightening rod is provided by a nut 36 anchored to proximal end segment 14, having a threaded portion 38 which connects to the proximal end of tightening element 32. Optionally, nut 36 can be molded into proximal segment 14 to anchor it.

In an exemplary embodiment of the invention, nut 36 also provides a connection 40 for coupling to an instrument for transforming the nail between its implantation and post-implantation states. Optionally, the nut proximal end 40 is a slotted or an Allen type connection.

Optionally, tightening element 32 includes cannulation 80 to allow insertion of nail 10 into the medullary canal over a guiding element, such as a K-wire.

The distal end segment 16 may optionally be cannulated or non-cannulated. FIGS. 2A and 3A illustrate a non-cannulated embodiment of distal segment 16 in which rod 32 is attached by a threaded connection 35 to lock tightening rod 32 and distal segment 16 together.

To accommodate an internal guide wire, distal segment includes cannulation 80, as shown in FIGS. 2B and 3B. Here, the distal end of rod 32 is press fitted into the cannulation at 34.

Cannulation 31 which carries rod 32 may have several alternative configurations. It may, for example, be collinear with the long axis of each segment. Alternatively, it may be eccentric to the long axis of the segments. Alternatively and/or additionally, the long axis of the cannulation is not parallel to the long axis of the segment. Some of the indicated alternatives may allow greater flexibility in location and orientation of the fixation element receiving holes.

As a further alternative, the cannulations in different segments may be differently oriented, again to better accommodate placement of the fixation element receiving holes.

Proximal end 28 of proximal section 14 optionally provides for coupling to an insertion tool described below. As illustrated in FIGS. 1A, 1B, 2A, 2B. 3A, and 3B, proximal end 28 of proximal section 14 may have an asymmetrical, for example, a “crown”-like shape, comprising one to more protrusions and/or notches 29, to allow for alignment of the insertion tool and to allow transfer of rotational movement. The proximal end 14 incorporates also a threaded bore 30, to allow for the connection of a tightening instrument to the nail 10.

As mentioned above, in the implantation state, the segments are relatively moveable to facilitate following the curvature of a bone during implantation. Good results are generally obtainable for configurations in which the range of angular motion between distal and proximal end surfaces of adjacent segments is in the range 1 to 8 Deg, preferably 2 to 5 Deg.

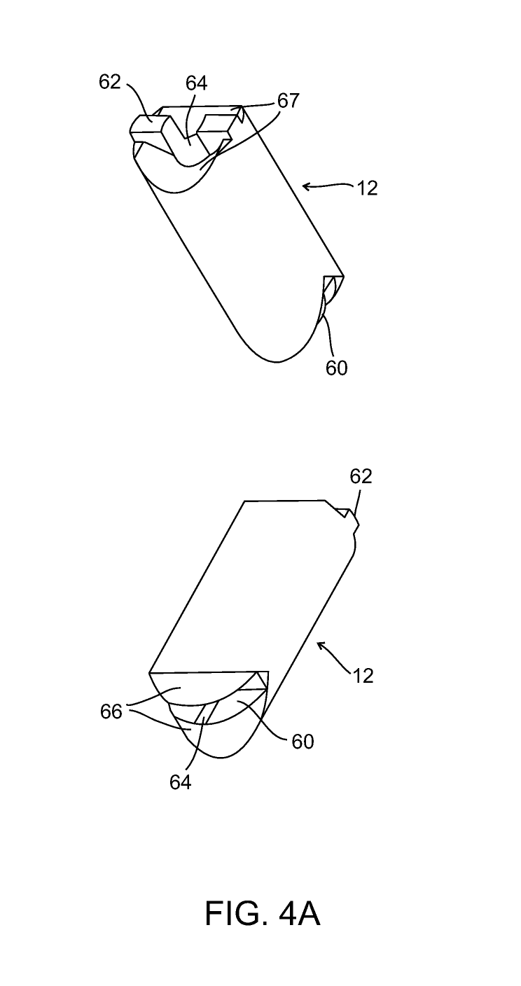

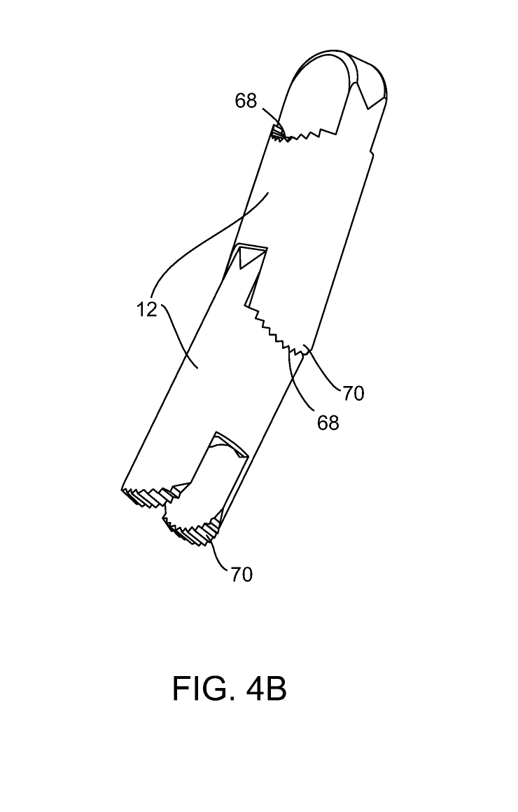

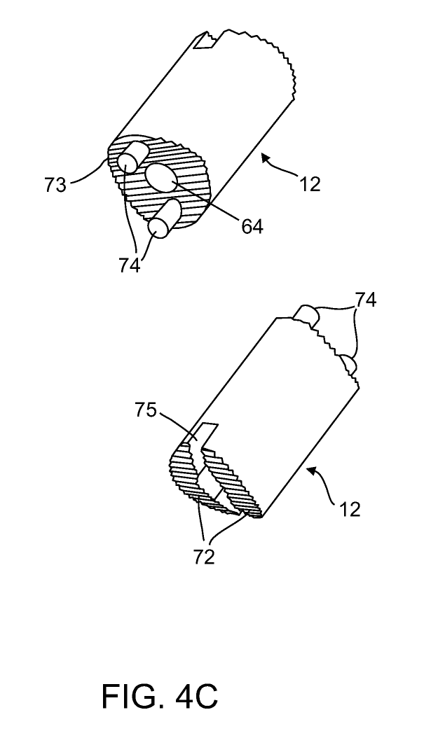

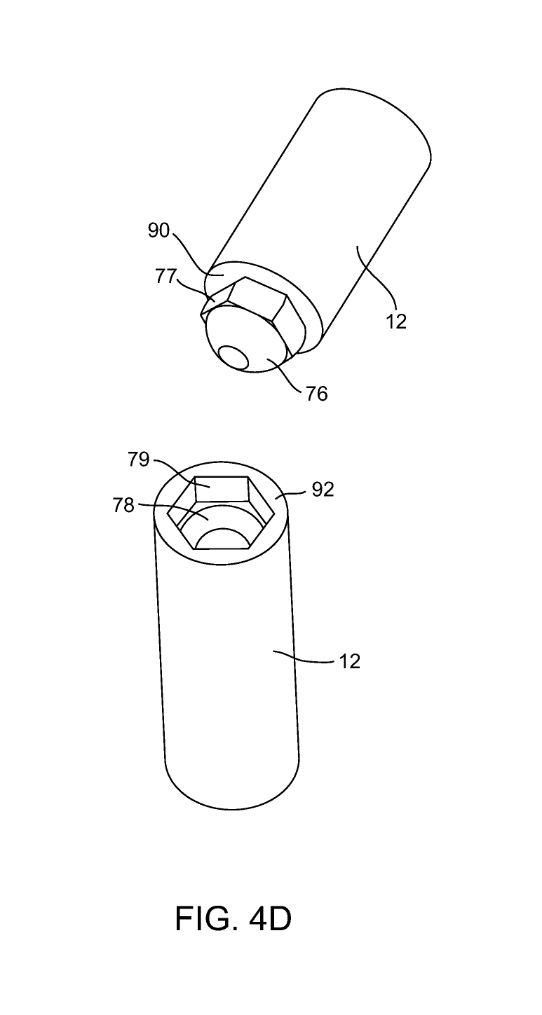

FIG. 4A is an illustration of an exemplary intermediate segment 12 which provides for a unidirectional flexibility of the nail.

In FIG. 4A, the segments are connected by a rib 62 on one end that engages with a slot 60 at the other end. Adjacent elements 60, 62 can be oriented at the same plane or in different planes (for example, at an angle of 90 degrees to each other). FIG. 4A also illustrates the segment cannulation 64. In the implantation state connection elements 60 and 62 provide for the transmission of rotational movement from one segment to the adjacent segment as well as longitudinal flexibility.

In the post-implantation state, surfaces 66, 67 are drawn together to lock the segments relative to each other. As will be understood, the orientation of the adjacent segments in the post-implantation state will be determined by the curvature of the medullary canal, to which the segments conform in the implantation state.

FIG. 4B is an illustration of 2 adjacent body segments 12 of a different exemplary design that provides unidirectional flexibility in the implantation state. Here, the upper and lower surfaces 68, 70 of each segment are roughened.

FIG. 4C is an illustration of an exemplary design of body segments 12, combining features of the body segments of FIGS. 4A and 4B, with different connection details 74, 75 designs that provide bidirectional flexibility. Here, projections, for example, pins 74 are configured to move freely within a recess, for example, a slot 75, when the nail is in the implantation state. Movement transversely to slot 75 is restricted by the difference between the diameters of pins 74 and the width of slot 75. The upper and lower surfaces 72, 73 of each segment are roughened to lock the segments in place when the nail is in the post-implantation state.

FIG. 4D is an illustration of a body segment 12 of yet another different exemplary design, providing for a transmission of rotational movement and for multidirectional flexibility of the nail. Connection details 76, 78 are rounded, to provide for multidirectional flexibility, while polygonal connection details 77, 79 provide for the ability to transmit rotational movement and or to help lock the segments together. Surfaces 90, 92 may be perpendicular to the long axes of body segments 12, or at any angle (ranging 0 to 90 or intermediate values) to the long axis of body segments 12. The angle of surfaces 90, 92 to the long axes of body segments 12 may vary between different body segments.

Although FIGS. 4A-4D refer to connections between intermediate sections 12, it should be understood that where no intermediate segments are employed, the configurations will be provided on the proximal and distal end segments 14 and 16.

FIGS. 5A and 5B are illustrations of two exemplary designs of a distal segment 16 that includes a cutting head 18.

FIG. 5A is an illustration of a distal segment 16 that is generally conical design, including one or more optional cutting surfaces 19. An optional cannulation 80 provides for insertion of the nail into the medullary canal over a guiding element, such as a K-wire, and/or for receiving the tightening element as previously described.

In FIG. 5B, an upper portion 18a includes one or more cutting surfaces 19, and/or also optional apertures 24 and 26 for receiving locking elements, for example, screws or K-wires, also as previously described. The distal tip 18b may be curved. As in the embodiment of FIG. 5A, a cannulation 80 provides for insertion of the nail into the medullary canal over a guiding element, such as a K-wire, and/or for receiving the tightening element.

FIG. 5C is an illustration of a distal segment 16 of yet another exemplary design. Here, distal end 18 is conical, to facilitate insertion and includes a cannulation 80. An optional hole 24 for locking element insertion is incorporated into the distal segment proximally of end 18.

Implant 10 may be provided as a single device or as part of a system provided as kit including an insertion tool, and a tightening tool which may optionally be part of the insertion tool. The kit may also include a cap configured to engage within the coupling to protect the coupling from bone in-growth, and/or to allow nail removal and one or more flexible sleeves covering at least part of the implant, to prevent tissue in-growth into the areas of connection of the device segments. Other conventional treatments such drugs may also be provided in association with the implant.

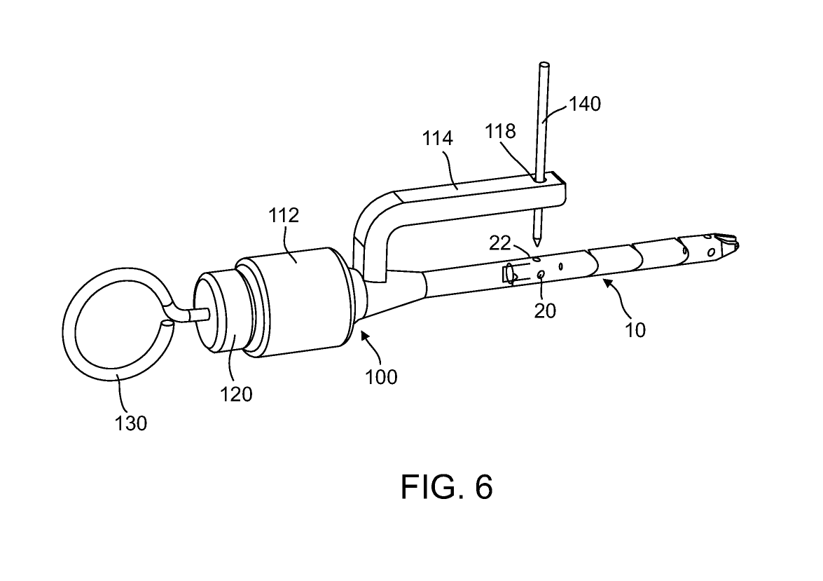

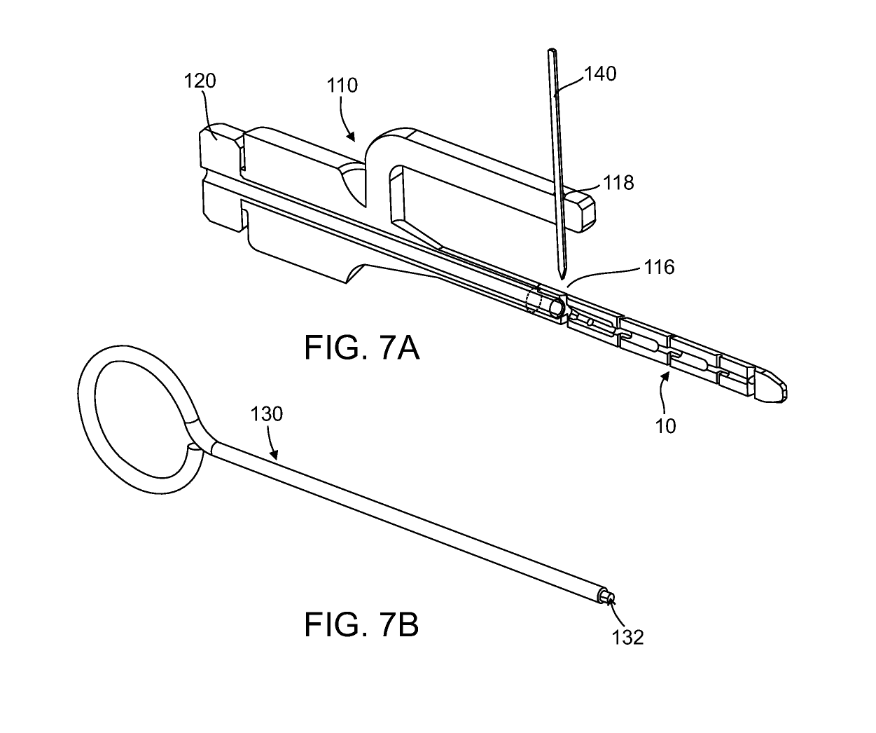

FIGS. 6, 7A, 7B and 8 are illustrations of nail 10 connected to an insertion assembly 100, and of the components of the insertion assembly. Insertion assembly 100 comprises insertion handle 110, connection tube 120, tightening tool 130, and guide sleeve 140.

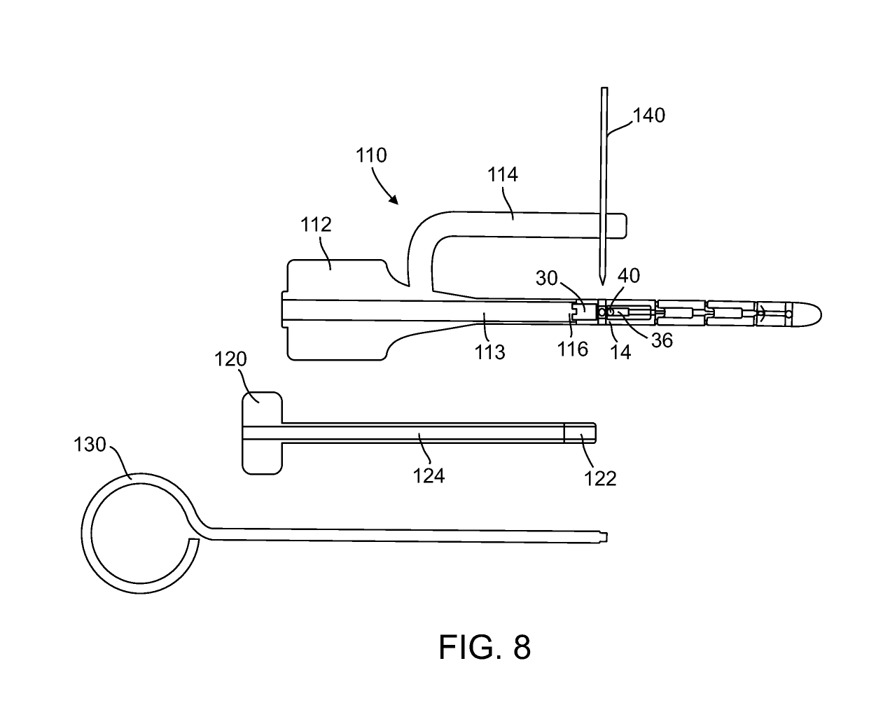

Insertion handle 110 has a body 112, which engages, at its distal end 116 (see FIGS. 6 and 8), with the proximal end 14 of the nail 10. The insertion handle is optionally cannulated at 124 to receive the tightening tool. The insertion handle incorporates, as an integral part or as a stand-alone item, an aiming device 114, to assist in insertion of locking elements (such as K-wire or screw) into nail 10 proximal segment holes 20, 22. Guide sleeve 140 can be inserted into hole(s) 118 in aiming device 114, to provide, for example for drilling holes for locking elements and as a guide for their implantation. Alternatively, the locking elements (for example, K-wire, etc.) can be inserted through hole 118 of aiming device 114 without the use of guide sleeve 140.

Aiming device 114 can, optionally, be moved around the perimeter of handle body 112, to be placed at different angles, against different holes for use during installation of the locking elements. Connection tube 120 is inserted into cannulation 113 of the insertion handle 120, and connects into bore 30 of the proximal segment 14 of the nail 10, into bore 30. The distal end 122 of connection tube 120 is threaded to provide for a connection to the nail 10 (by threading it into bore 30).

Tightening tool 130 is inserted into cannulation 124 of the connection tube 120. The distal end 132 of the tightening tool 130 is shaped to conform to the shape of the proximal end 40 of nut 36 located at the proximal end 14 of the nail 10.

After reduction the nail is transformed into its rigid mode. In order to make the nail rigid, the tightening tool 130 is inserted via the cannulation 124 in the connection tube 120 until its distal end 132 engages with the proximal end 40 of nut 36. The tightening tool is rotated clockwise. This causes the tightening element 32 to be threaded over the distal section 38 of nut 36, thus pulling the nail segments 12, 16 towards the most proximal segment 14 and tightening them together. Alternatively, nail 10 may be designed such that tightening tool 130 will be rotated counterclockwise in order to make the nail rigid. The threads are made fine enough that friction helps resist loosening of the rod in the post implantation state.

Optionally, insertion tool 110 and tightening tool 130 may be delivered pre-attached to implant 10, and may be designed for disposal after use. Alternatively, one or more of the tools may be designed for re-use.

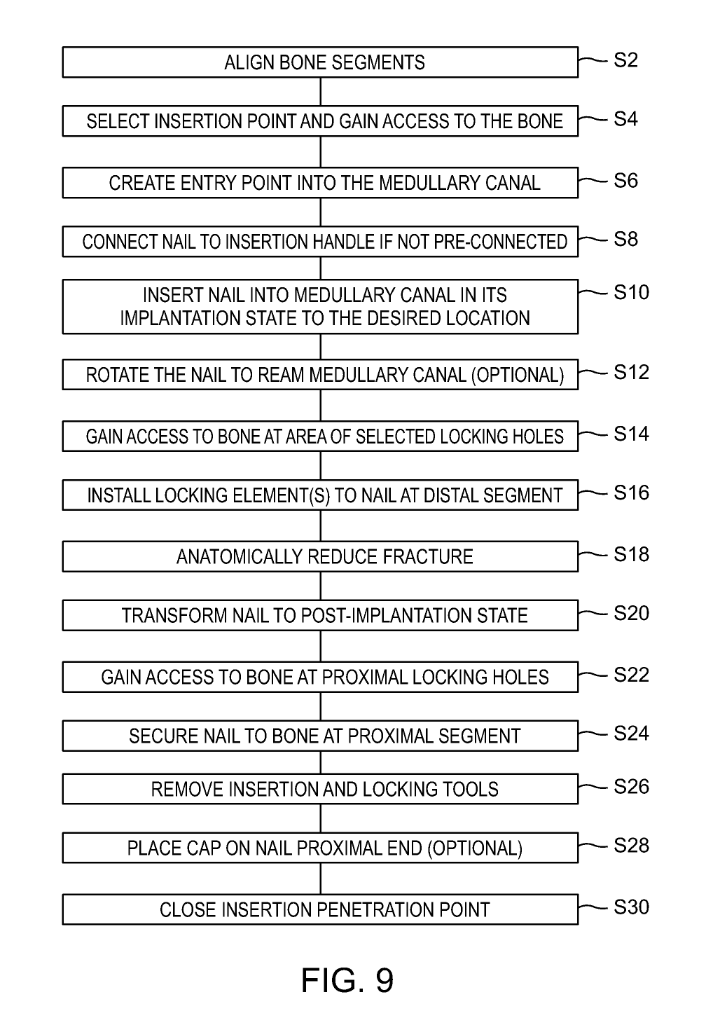

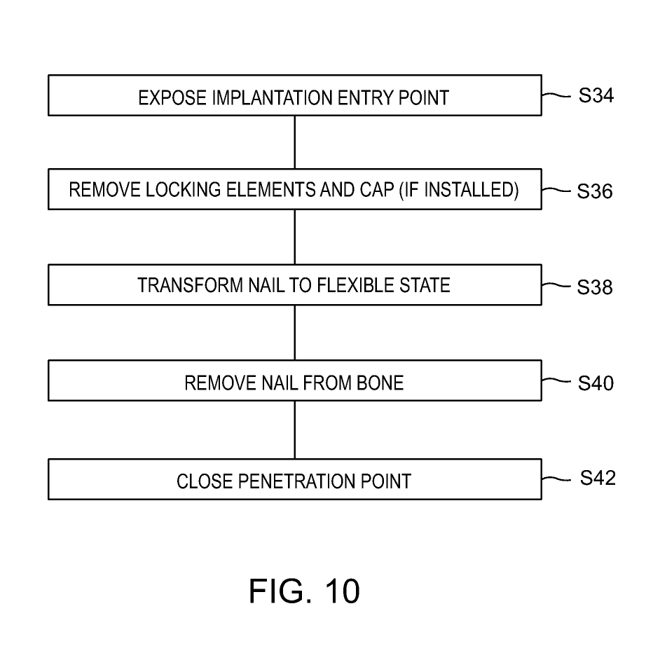

The way in which the present invention may be used is believed to be clear to persons skilled in the art, from the foregoing descriptions. However, for illustration purposes only, FIGS. 9 and 10 provide flow charts showing some methods of installation and removal according to some embodiments of the present invention.

Referring to FIG. 9, at S2 the fractured bone segments are aligned. At S4, an insertion point is selected and access to the bone is gained in a normal manner, followed by bone penetration, to create an entry point into the medullary canal. Choice of the location for entry is enhanced by the fact that the implant segments can move and are short enough to permit insertion from middle of bone or other convenient location, rather than end

At S6, an entry point into the medullary canal is created in a conventional manner.

At S8, the insertion handle is connected to the nail using connection tube 120 if not pre-connected or previously connected in preparation for the surgery.

At S10, nail 10 is inserted into the medullary canal in its flexible implantation state and advanced through the medullary canal to reach its desired location using insertion tool 110. Where there is one degree of freedom of motion of the segments, the bending direction of device is aligned with the curvature of the bone. Where the segment interfaces permit two degrees of freedom of motion, the surgeon will have greater flexibility in orienting the implant for insertion.

At S12, if distal tip 18 of the distal segment 16 is configured as a cutting head, the nail may optionally be rotated around its long axis to ream the medullary canal during nail insertion.

At S14, access is gained to the bone at the area of selected distal locking holes to avoid potential damage to nearby organs or tissue, and at S16, one or more locking elements, such as K-wires, are inserted to secure the nail to the bone at its distal segment 16. Installation of the K-wire(s) is performed using free hand technique.

At S18, the fracture is optionally anatomically reduced. During reduction, if distal locking elements are inserted, the distal section of the bone can be pulled towards the proximal section of the bone, using axial and/or torsional movements. The radiolucency of the segments in the vicinity of the fracture and the radiopaque markings help the surgeon perform the insertion and the reduction by allowing radiographic visualization.

At S20, after reduction the nail is transformed into its post-implantation state using tightening tool 130 inserted via the cannulation 124 in the connection tube 120 until its distal end 132 engages with the proximal end 40 of nut 36. The tightening tool is then rotated. This causes the tightening element 32 to be threaded over the distal section 38 of nut 36, thus pulling the nail segments 12, 16 towards the most proximal segment 14 and tightening them together. If necessary, the nail can be partially tightened during reduction,

Then, at S22, access is gained to the bone at the area of the proximal locking holes, and at S24, the nail is secured to the bone at the proximal segment 14 through a desired combination of the available holes, K-wire(s) insertion to the proximal segment 14 is performed either free hand or using the aiming device 114, and, optionally, guide sleeve 140. Locking the proximal segment to the bone helps assure that the post-implantation state of the nail is maintained by preventing separation of the segments.

At S26, the insertion and locking tools are removed, and at S28, the cap is optionally attached to the proximal end of the nail.

At S30, the insertion penetration point is closed.

Thereafter, the installation tools are optionally discarded, or prepared for subsequent use if so designed.

FIG. 10 shows the process of implant removal according to some embodiments.

At S34, the implantation entry point is exposed, and the locking elements and cap are removed if used.

At S36, the nail is transformed to its flexible state by turning the rod in the reverse direction from that used to tighten the implant.

At S38, the nail is pulled out of the bone, and at S40, the penetration points are closed.

Various features of devices and methods have been described. It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art, which would occur to persons skilled in the art upon reading the foregoing description.

The scope of the invention also covers machines for creating the apparatus described herein. In addition, the scope of the invention also includes methods of using, constructing, calibrating and/or maintaining the apparatus described herein.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

1. An intramedullary implant comprising:
an elongated structure including a proximal end segment and a distal end segment;
one or more intermediate segments between the proximal and distal end segments; and
a coupling arrangement including a tightening element that extends between the proximal and distal segments,
wherein the coupling arrangement provides a first implantation state in which the segments are relatively moveable with respect to each other, to allow conformity to curvature of a bone, and a second post-implantation state in which the segments are geometrically locked together to provide a substantially rigid structure that substantially follows the curvature of the bone which it assumed during implantation;
wherein said proximal and distal segments comprise holes configured for receiving locking elements to fix the implanted nail in the bone; said holes arranged such as to provide access from at least four directions; and
wherein at least a portion of said segments comprise composite material.

2. An implant according to claim 1, wherein one or more of the surfaces of adjacent segments include surface features configured to help lock the adjacent surface together when the device is in the rigid state, whereby the segments remain substantially in the relative positions assumed in the flexible state.

3. An implant according to claim 1, wherein the distal segment includes a cutting surface configured to allow reaming of the intramedullary canal during insertion.

4. An implant according to any of claim 1, wherein said holes are located on a plane transverse to a long axis of said implant.

5. An implant according to claim 1, wherein the tightening element extends between the proximal and distal end segments through cannulations in the segments, and is operable to draw the distal and proximal end segments toward each other during transformation from the implantation state to the post-implantation state, and to separate the distal and proximal end segments during transformation from to the implantation state.

6. An implant according to claim 5, wherein the tightening element is comprised of a rod that extends through cannulations in the segments.

7. (canceled)

8. An implant according to claims 1, wherein the tightening element is made of longitudinally oriented fibers or interwoven cable, or fibers embedded within a matrix made of implantable material.

9. (canceled)

10. An implant according to claim 6, wherein the rod is cannulated and is configured to receive a guide element.

11. An implant according to any of claims 6, wherein the rod is fixedly connected to the distal end segment, and is threadedly connected to the proximal end segment.

12-13. (canceled)

14. An implant according to claim 1, wherein the holes are positioned externally to the tightening element.

15. An implant according to claim 1, wherein an interface between adjacent segments of the device allows about two degrees of freedom when the implant is in the implantation state.

16. An implant according to claim 2, wherein the surface features are one or more of (a) surface treatment to increase friction between the surfaces in the post-implantation state; (b) sharp protrusions made of implantable materials that are harder than the material of which the segment is made; and (c) projections that engage with recesses in the adjacent surface.

17. (canceled)

18. An implant according to claim 16, wherein portions of the projections and the recesses are configured to form a ball and socket connection, and further portions are configured to lock together in the post-implantation state.

19. An implant according to claim 1, further including a threaded coupling element for connection of the implant to a threaded portion of an insertion and/or a tightening tool.

20. An implant according to claim 1, wherein the tightening element includes an asymmetrical coupling at its proximal end configured to receive a tightening tool in a predetermined orientation and to allow transfer of rotational movement.

21. An implant according to claim 1, wherein said implant is at least partially formed of a radiolucent material.

22. A long-bone fixation implant comprising a segmented intramedullary nail at least partially formed of a radiolucent material;
wherein the intramedullary nail comprises an elongated structure including a proximal end segment and a distal end segment; and
a coupling arrangement including a tightening element that extends between the proximal and distal segments, wherein the coupling arrangement provides a first implantation state in which the proximal and distal segments are relatively moveable, to allow conformity to curvature of a bone, and a second post-implantation state in which the segments are locked together to provide a substantially rigid structure that substantially follows the curvature of the bone which it assumed during implantation.

23. An implant according to claim 22, wherein the nail is formed of a radiolucent ceramic or of a polymeric material, the polymeric material comprising a matrix including at least one of PEEK, PEKK, PAEK, and carbon reinforcing fibers.

24-25. (canceled)

26. An implant according to claim 22, wherein the intramedullary nail includes one or more intermediate segments, and wherein the intermediate segments are formed of a radiolucent material and the distal end segment is formed of metal.

27. (canceled)

28. An implant according to claim 22, further including one or more radiopaque markings to allow visualization of the implant by radiographic imaging.

29. An implant according to claim 28, wherein the radiopaque markings are comprised of one or more of (a) elongated markers extending parallel to a long axis of one or more of the segments, (b) one or more markers defining extending around the periphery of one or more of the segments, and (c) markings positioned to show the positions of holes for receiving fixation elements;
wherein the markings are wires formed of at least one of tantalum, platinum, gold and tungsten.

30. (canceled)

31. An implant according to claim 19, further including at least one of a cap and one or more flexible sleeves, said cap and sleeves configured to protect at least a part of the implant from bone in-growth, and/or to facilitate removal of the implant.

32-41. (canceled)
